(19) United States
(12) Patent Application Publication
(10) Pub. No.: US 2014/0206928 A1
(43) Pub. Date: Jul. 24, 2014

(54) EXFOLIATING FOOT COVERING THAT COVERS TOES

(71) Applicant: Jennifer Kay Snider-Tornetta, Sayreville, NJ (US)

(72) Inventor: Jennifer Kay Snider-Tornetta, Sayreville, NJ (US)

(21) Appl. No.: 13/866,365

(22) Filed: Apr. 19, 2013

Related U.S. Application Data
(63) Continuation-in-part of application No. 13/826,915, filed on Mar. 14, 2013.
(60) Provisional application No. 61/755,037, filed on Jan. 22, 2013.

(51) Int. Cl. A61B 17/54; A61N 2/00; A61H 7/00

(52) U.S. Cl. CPC A61B 17/54; A61H 7/004; A61N 2/002
USPC 600/15; 601/46

(57) ABSTRACT

A sock has an inner material or surface that causes skin on portions of the human foot, which are covered by the sock, to be exfoliated while the sock is worn. Massaging elements and/or magnetic elements on the sock causes the human foot to be massaged and/or be subjected to a magnetic field, respectively when the sock is worn. The sock includes an upper cuff portion that enables the sock to fit snuggly around the portions of the human foot, which are covered by sock. The upper cuff portion includes a string and/or an elastic material that enables the upper cuff portion to fit snugly. The massaging elements and/or magnetic elements may be located along an interior and/or exterior portion of the sock and/or may be located so that they protrude inside the sock and also outside sock. A toe portion of the sock covers an area around the toes.

### CROSS-REFERENCE TO RELATED APPLICATIONS/INCORPORATION BY REFERENCE

This application is a continuation in part of U.S. application Ser. No. 13/826,915, which was filed on Mar. 14, 2013.

This application makes reference to, claims priority to and claims the benefit of U.S. Provisional Application Ser. No. 61/755,037, which was filed on Jan. 22, 2013.

Each of the above referenced applications is hereby incorporated herein by reference in its entirety.

### FIELD OF THE INVENTION

Certain embodiments of the invention relate to a cosmetic foot apparatus. More specifically, certain embodiments of the invention relate to a method and apparatus for an exfoliating foot covering that covers toes.

### BACKGROUND OF THE INVENTION

Many spa, salon or in-home pedicures are being done every day; consumer's spend a great deal of time and pay a great deal of money for pedicures in many cases once the pedicure is complete, preserving the full results of this service are not available. Specifically, certain pedicure services generally exhibit characteristics that exfoliate parts of the human foot. For example, certain pedicure services may incorporate a rubbing of the heel and toe portion of the human foot by using an object that causes friction, thereby exfoliating the dead skin cells off of the foot. For example, certain pedicure services may wash the user's foot using a granular cosmetic process in order to remove dead cells from the skin's surface. Often times, harsh chemicals and dangerous mechanical devises are used for this process and puts the user in unnecessary harm.

Further limitations and disadvantages of conventional and traditional approaches will become apparent to one of skill in the art, through comparison of such systems with some aspects of the present invention as set forth in the remainder of the present application with reference to the drawings.

### BRIEF SUMMARY OF THE INVENTION

A system and/or method is provided for an exfoliating foot covering that covers toes, substantially as shown in and/or described in connection with at least one of the figures, as set forth more completely in the claims.

These and other advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 is a diagram illustrating an exemplary exfoliating sock being worn on a human foot, in accordance with an embodiment of the invention.

FIG. 2 is a diagram illustrating an exemplary exfoliating sock being worn on a human foot, in accordance with an embodiment of the invention.

FIG. 3 is a diagram illustrating a top frontal view of an exemplary exfoliating sock, in accordance with an embodiment of the invention.

FIG. 4 is a diagram illustrating a top view of an exemplary exfoliating sock, in accordance with an embodiment of the invention.

FIG. 5 is a diagram illustrating a top view of an exemplary exfoliating sock comprising a fastener, in accordance with an embodiment of the invention.

FIG. 6 is a diagram illustrating a top view of an exemplary exfoliating sock comprising an angled fastener, in accordance with an embodiment of the invention.

FIG. 7 is a diagram illustrating a top view of an exemplary exfoliating sock comprising a fastener, in accordance with an embodiment of the invention.

FIG. 8 is a diagram illustrating a top view of an exemplary exfoliating sock comprising an angled fastener, in accordance with an embodiment of the invention.

FIG. 9A is a diagram illustrating a side view of an exemplary exfoliating sock that covers the toes, in accordance with an embodiment of the invention.

FIG. 9B is a diagram illustrating a side view of an exemplary exfoliating sock that covers the toes, in accordance with an embodiment of the invention.

FIG. 10A is a diagram illustrating a side view of an exemplary exfoliating sock that covers the toes and comprises massaging elements, in accordance with an embodiment of the invention.

FIG. 10B is a diagram illustrating a side view of an exemplary exfoliating sock that covers the toes and comprises massaging elements, in accordance with an embodiment of the invention.

FIG. 10C is a diagram illustrating a side view of an exemplary exfoliating sock that covers the toes and comprises massaging elements, in accordance with an embodiment of the invention.

FIG. 10D is a diagram illustrating a side view of an exemplary exfoliating sock that covers the toes and comprises massaging elements, in accordance with an embodiment of the invention.

FIG. 11A is a diagram illustrating a side view of an exemplary exfoliating sock that covers the toes and comprises massaging elements, in accordance with an embodiment of the invention.

FIG. 11B is a diagram illustrating a side view of an exemplary exfoliating sock that covers the toes and comprises massaging elements, in accordance with an embodiment of the invention.

### DETAILED DESCRIPTION OF THE INVENTION

Certain embodiments of the invention may be found in a method and system for an exfoliating foot covering that covers toes. Various aspects of the invention may comprise a foot covering to be worn on a human foot. The foot covering comprises a sock that covers the human foot when it is worn. The sock may comprise an inner material or surface that causes skin on portions of the human foot, which are covered by the sock, to be exfoliated while the sock is worn on the human foot. The sock also comprises one or more massaging elements and/or one or more magnetic elements that cause the human foot to be massaged and/or be subjected to a magnetic field, respectively. Concurrent with the exfoliation, massaging elements are operable to massage at least a portion of the human foot that is within proximity of the massaging elements, and/or the magnetic elements are operable to provide a magnetic field to at least a portion of the human foot that is within proximity of the magnetic elements. The sock may also comprise an upper cuff portion that enables the sock to fit snuggly around the portions of the human foot, which are covered by said sock. The upper cuff portion may comprise a string and/or an elastic material that enables the upper cuff portion of the sock to fit snugly.

In some embodiments of the invention, the massaging elements and/or the magnetic elements may be located along an interior or inside portion of the sock and/or an exterior or outer portion of the sock. In some embodiments of the invention, the massaging elements and/or the magnetic elements are located so that they protrude in an interior portion of the sock and an exterior portion of the sock. The sock, which covers portions of the human foot, comprises an upper cuff portion, a heel portion, an instep portion, a sole portion and a toe portion. The upper cuff portion may cover at least an area within the vicinity of an ankle of the human foot. The heel portion may cover at least an area around a heel of said human foot. The instep portion covers at least an area around an instep of the human foot. The sole portion may cover at least an area around a sole of the human foot. The toe portion covers at least an area around toes of the human foot. In some embodiments of the invention, the toe portion of the sock may comprise a non-abrasive material. In some embodiments of the invention, different portions of the interior or inner material of the sock may comprise varying grades of abrasiveness. The inner material of the sock is operable to gently move against the skin on the portions of the human foot, which are covered by the sock, to cause the skin to be exfoliated while the foot is being massaged and/or being subjected to a magnetic field. The material of the sock may be breathable, antifungal, antibacterial, light weight, porous material and/or quick drying. The material for the massaging elements and the magnetic elements may be antifungal, antibacterial and/or light weight.

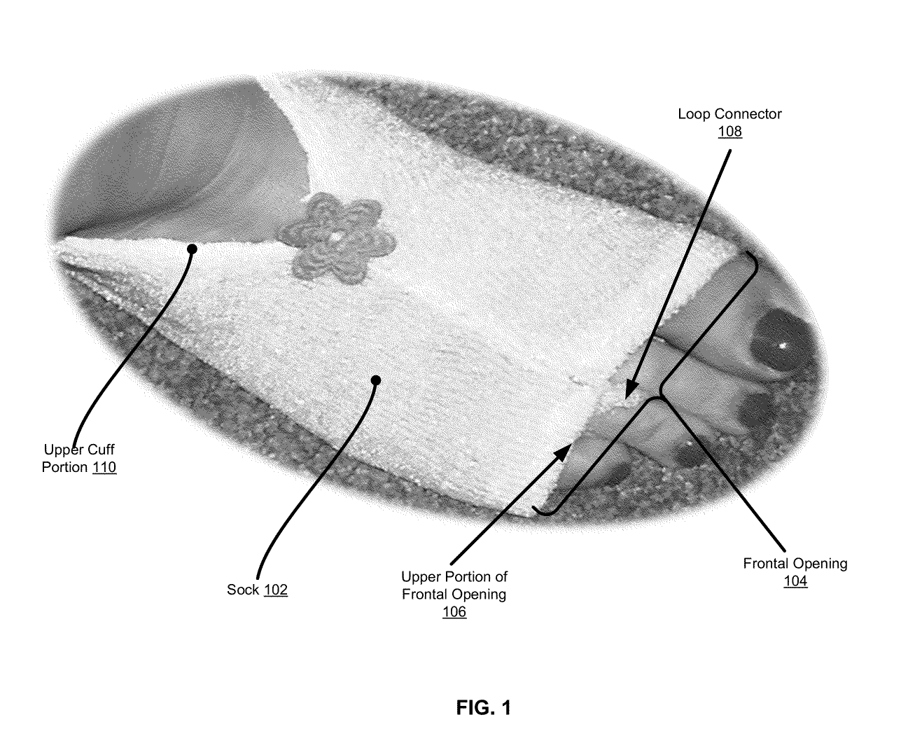

FIG. 1 is a diagram illustrating an exemplary exfoliating sock being worn on a human foot, in accordance with an embodiment of the invention. Referring to FIG. 1, there is shown a sock 102, a frontal opening 104, an upper portion of the frontal opening 106, a loop connector 108, and an upper cuff portion 110. The sock 102 is shown being worn on a foot. The toes on the foot are not covered and thus remain exposed by the sock 102.

The sock 102 is operable to be worn on the human foot. The sock 102 may comprise a frontal opening 104 that causes the toes of the human foot to be exposed. The sock 102 may comprise an inner material or surface that causes skin on portions of the human foot, which are covered by the sock 102, to be exfoliated while the sock is being worn on the human foot. The exfoliation may result from the skin of the human foot rubbing against the abrasive material of the inner portion of the sock 102.

The frontal opening 104 in the sock 102 allows the toes of the human foot to be exposed. The frontal opening 104 allows the sock to be worn so that the abrasive material of the sock does not in any way damage the natural state of the toenails and/or any decorations and/or material, which may be on the toenails. The frontal opening 104 may comprise an upper portion and a lower portion. The upper portion may be referred to as the upper portion of the frontal opening 106 and the lower portion may be referred to as the lower portion of the frontal opening.

The loop connector 108 may be arranged so that it couples the upper portion of the frontal opening 106 to the lower portion of the frontal opening. The loop connector 108 may be operable to prevent slippage of the sock 102 while it is on the human foot. The loop connector 108 may also be operable to prevent the sock 102 from rolling up while it is on the human foot.

The upper cuff portion 110 of the sock 102 may be configured so that it rests around an ankle area of the human foot when the sock is properly placed on the human foot. The upper cuff portion 110 of the sock 102 may be configured so that it is at, below or above the ankle of the human foot. In order to put on the sock 102, the toes are placed in the circumferential opening of the upper cuff portion 110 of the sock 102 and the human foot is guided through the upper cuff portion 110 of the sock 102. The human foot is guided through the circumferential opening of the upper cuff portion 110 of the sock 102 until the toes are adequately exposed and the loop connector 108 is between two toes as illustrated in FIG. 1. When the sock is properly on the human foot, the heel of the human foot rests in a heel portion of the sock 102. The human foot may be guided through the circumferential opening of the upper cuff portion 110 of the sock 102 and through the remaining portion of the sock without the toes touching the inner material of the sock 102. This may prevent marring or damaging of the toenails. In instances where, for example, toenail polish may be on the toenails, the sock 102 may prevent the toenail polish from smudging, smearing or other damage.

In operation, the sock 102 is operable to perform a gentle rubbing action against the skin of the human foot as the human foot moves. The gentle rubbing action may be performed in a natural, safe and secure manner so as not to cause any damage to the skin of the human foot. The sock 102 covers the heel of the human foot and extends to the forefront of the human foot. The sock 102 cuffs the foot, for example, around an area of the ankle and starts from the back heel and extends forward towards the toe area and frames the forefront of the human foot just before the toe portion. The sock 102, when properly placed on the human foot, leaves the toes of the human foot exposed. This, for example, prevents any damage to the toenails and/or any material thereon.

The sock 102 may be used wet or dry, while sleeping in bed, performing chores and/or performing showering activities, and enhances the look of the human foot. When lying in bed, doing chores and/or standing in a shower, for example, the natural body movements causes friction against a surface and has the ability to exfoliate when rubbed against the surface. Accordingly, the natural body movements causes friction when the areas of the human foot that are covered by the sock 102 rub against the inner abrasive material of the sock 102 and this exfoliates the skin on the areas of the human foot that are covered by the sock 102.

The sock 102 may be formed from textured material, such as synthetic nylon fiber, which creates friction and thereby, removing dead skin cells from the human foot. Furthermore, the sock 102 may incorporate or comprise materials that are specifically selected for particular environmental conditions, such as water or dry conditions. Material elements incorporated into sock 102 are generally selected to impart various aesthetic and functional characteristics comprising, for example, natural beauty, exfoliating properties, friction, water and/or non-water assistance, durability, and slow decaying. The exfoliating sock 102 may comprise, for example, a lightweight mesh material, synthetic nylon fiber, or some other lightweight and friction inducing material designed to naturally and safely remove dead skin cells while the sock 102 is worn during, for example, sleeping, relaxing or showering. The sock 102 may utilize material that may be woven together for strength, durability, slow decaying and stretching qualities. The sock 102 may comprise material that dries quickly, easily and doesn't breed bacteria. The sock 102 may comprise high elasticity yarn, which may be woven together, to implement a multistage exfoliating knit that may adjust the force of the elastic threads and the resulting exfoliating qualities. The natural movements of the person wearing the sock 102 promote blood circulation, thereby minimizing and preventing skin disease. Accordingly, the exfoliating sock 102 may be operable to provide variable exfoliating qualities on various parts of the human foot.

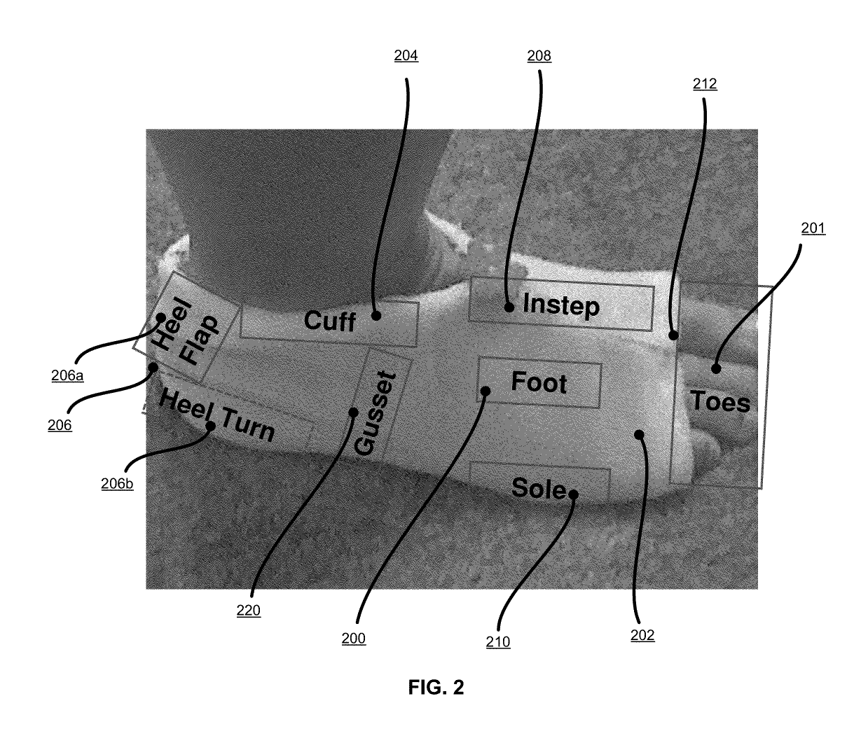

FIG. 2 is a diagram illustrating an exemplary exfoliating sock being worn on a human foot, in accordance with an embodiment of the invention. Referring to FIG. 2, there is shown a sock 202 being worn on a human foot 200 with the toes 201 of the human foot being exposed. The sock 202 comprises an upper cuff portion 204, a heel portion 206, an instep portion 208, a sole portion 210, a frontal opening 212 and a gusset portion 220. The heel portion 206 may comprise a heel flap portion 206a and a heel turn portion 206b.

The sock 202 may comprise an opening at the upper cuff portion 204 and top of the back heel section in order for the wearer of the sock 202 to easily insert the human foot 200 into the sock 202 and encase the human foot 200 from heel 206 to frontal opening 212. The area around the heel 206 extends upwards towards the anklebone and slightly cascades down on the top of the human foot 200 to enable the wearer to easily insert the foot 200. The body of the sock 202, which may also comprise the instep portion 208, may comprise elastic qualities from the heel portion 206 toward the forefront portion or the frontal opening 212 so that most wearers regardless of their foot size may wear the sock 202. The body of the sock 202 does not contain a toe portion and ends at the frontal opening 212 just before the toe area of the human foot 200. This prevents decorations on the toenails such as toenail polish from being damaged or smudged due to the abrasive material of the inner portion of the sock 202.

The upper cuff portion 204 is the uppermost part of the sock 202. The human foot 200 is inserted into circumferential opening of the upper cuff portion 204 of the sock 202 and the material of the sock 202 wraps around the left, right and back side of the ankle of the foot. The upper cuff portion 204 has elasticity to stretch the sock 202 around the wearer's ankle as well as to hold the sock 202 in place on the wearer's foot. The upper cuff portion 204 cascades down the center of the top of the wearer's foot in a triangular manner and may comprise a ribbing around the cuff, which may, for example, be less than 1 inch in diameter.

The upper cuff portion 204 may cover at least an area around an upper portion of an ankle of the human foot 200 when the sock 202 is properly placed on the human foot 200. The elasticity of the upper cuff portion 204 of the sock 202 enables the sock 202 to fit snuggly around the portions of the human foot 200, which are covered by the sock 202. The circumference of the upper cuff portion 204 may comprise a mechanism such as a string and/or an elastic material that enables the upper cuff portion 204 of the sock to fit snugly around the human foot 200. Other mechanisms and/or materials may be utilized without departing from the spirit and scope of the various embodiments of the invention.

The heel portion 206 may cover at least an area around a heel of the human foot 200 when the sock 202 is properly placed on the human foot 200. The heel portion 206 may comprise the heel flap portion 206a and the heel turn 206b portion.

The heel flap portion 206a comprises the area of the sock 202 that covers the heel 206 when the sock 202 is properly fitted on the human foot 200. The heel flap portion 206a may comprise a very strong material in order to prevent this area of the sock 202 from being worn out. In this regard, the heel flap portion 206a may comprise a thicker material than other parts of the sock 202. The heel flap portion 206a may comprise one area where a great deal of exfoliation occurs.

The heel turn 206b portion is the part of the sock 202 that cradles the heel 206 and bottom of the foot when the sock 202 is properly fitted on the human foot 200. The heel turn 206b portion comprises a very strong material in order to prevent this area of the sock 202 from being worn out. In this regard, the heel turn portion 206b may comprise a thicker material than other parts of the sock 102. The heel turn portion 206b may comprise another area where a great deal of exfoliation occurs

The instep portion 208 forms part of the body of the sock 202. The instep portion 208 of the sock 202 may be viewed as the part of the sock 202 starting from where the gusset 220 ends and the toe 201 begins. The instep portion 208 covers at least an area around an instep of the human foot 200 when the sock 202 is properly placed on the human foot 200. The length of the instep portion 208 may be generally determined by measuring the wearer's foot from the back of the heel 202 to the tip of the toe 201 and subtracting 1.5″ to 2″ from that measurement. This is because the sock 202 does not have a toe portion since the toes 201 are exposed. The instep portion 208 may comprise elastic properties, which may run from the heel turn portion 206b to the forefront of the foot.

The sole portion 210 covers at least an area around a sole of the human foot 200 when the sock 202 is properly placed on the human foot 200.

The frontal opening 212 in the sock 202 causes the toes 201 of the human foot 200 to be exposed when the sock 202 is properly placed on the human foot 200. A circumference of the frontal opening 212 of the sock 202 may comprise a mechanism such as a string and/or an elastic material that enables the frontal opening 212 to be expanded. Other mechanisms and/or materials may be utilized without departing from the spirit and scope of the various embodiments of the invention. The string and/or elastic material at the circumference of the frontal opening 212 may enable the frontal opening 212 to fit snugly around the human foot 200.

The gusset 220 connects the heel flap portion 206a and heel turn portion 206b of the sock 202 to the foot of the sock 202. The gusset 220 goes from the right side of the human foot 200, around the sole 210, and up the left side of the foot 200. The gusset 220 does not go across the top of the human foot 200 because the upper cuff portion 204 extends down slightly on the top of the human foot 200.

In operation, an inner material or surface of the sock 202 may cause the skin on portions of the human foot 200, which are covered by the sock 202, to be exfoliated while the sock 202 is being worn on the human foot 200. In one embodiment of the invention, the inner material of the sock 202 may comprise the same grade of abrasiveness throughout the sock 202. In another embodiment of the invention, different portions of the inner material of the sock 202 may comprise varying grades of abrasiveness. In this regard, material with greater abrasiveness may be placed at those areas of the sock 202 where the foot 200 may need more exfoliation and material with lesser abrasiveness may be placed at those areas of the sock 202 where the foot 200 may need less exfoliation. In some areas of the sock 202, the material may be raised and/or contoured to match the contour of the human foot 200. The inner material of the sock 202 is operable to gently move against the skin on those portions of the human foot 200, which are covered by the sock 202, in order to cause the skin to be exfoliated. The material that is utilized to make the sock 200 possesses one or more exemplary properties comprising breathable, antifungal, antibacterial, lightweight, elastic, porous material and quick drying.

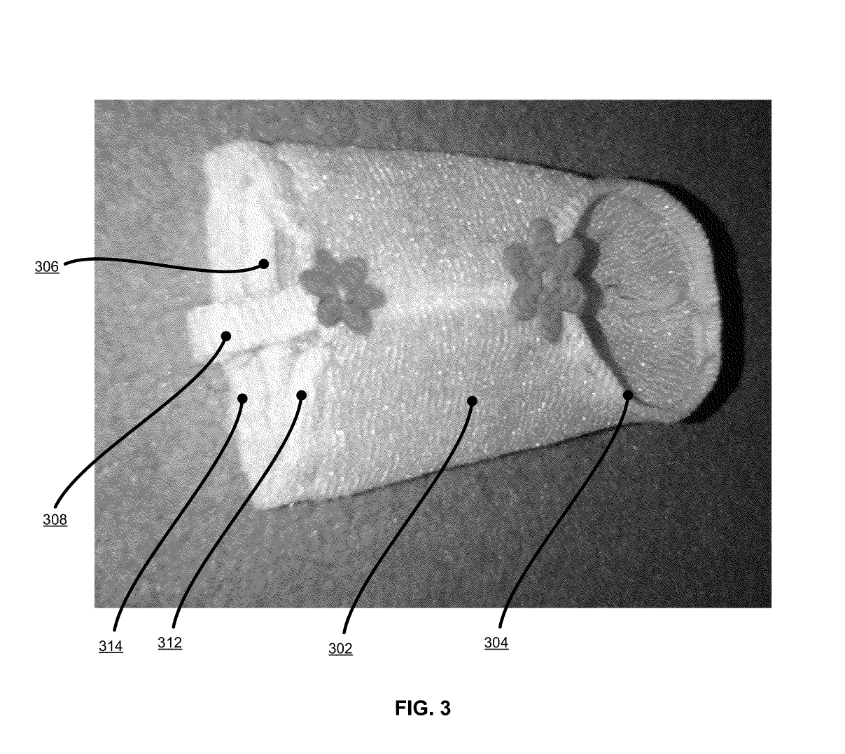

Various exemplary embodiments of the invention may comprise a foot covering to be worn on a human foot 200. With reference to FIG. 2, the foot covering comprises a sock 202 that covers the human foot 200 when the sock 202 is worn. The sock 202 may be characterized by a frontal opening 212 that causes the toes 201 of the human foot 200 to be exposed. The sock 202 may comprise an inner material or surface that causes skin on portions of the human foot 200, which are covered by the sock 202, to be exfoliated while the sock 202 is being worn on the human foot 200. The sock 202 (302 in FIG. 3) may comprise one or more loop connectors 308, (FIG. 3) each of which couples an upper portion of the frontal opening 312 (FIG. 3) to the lower portion of the frontal opening 314 (FIG. 3). In this regard, the one or more loop connectors 308 (FIG. 3) are arranged so that they may fit between at least two toes 201 on the human foot 200. An upper cuff portion 204 of the sock 202 enables the sock 202 to fit snuggly around the portions of the human foot 200, which are covered by the sock 202. The circumference of the upper cuff portion 204 may comprise a mechanism such as a string and/or an elastic material that enables the upper cuff portion 204 of the sock to fit snugly around the human foot 200.

A circumference of the frontal opening 212 of the sock 202 may comprise a mechanism such as a string and/or an elastic material that enables the frontal opening 212 to be expanded. The string and/or an elastic material at the circumference of the frontal opening 212 may enable the frontal opening 212 to fit snugly around the human foot 200. The portions of the human foot 200, which are covered by the sock 202, may comprise an upper cuff portion 204 a heel portion 206, an instep portion 208 and a sole portion 210. The upper cuff portion 202 may cover at least an area around an upper portion of an ankle of the human foot 200. The heel portion 206 may cover at least an area around a heel of the human foot 200. The instep portion 208 covers at least an area around an instep of the human foot 200 and the sole portion 210 covers at least an area around a sole of the human foot 200. The inner material of the sock 202 may comprise the same grade of abrasiveness. Alternatively, different portions of the inner material of the sock 202 may comprise varying grades of abrasiveness. The inner material of the sock 202 is operable to gently move against the skin on those portions of the human foot 200, which are covered by the sock 202, in order to cause the skin to be exfoliated. The material that is utilized to make the sock 200 possesses one or more exemplary properties comprising breathable, antifungal, antibacterial, lightweight, porous material and quick drying.

FIG. 3 is a diagram illustrating a top frontal view of an exemplary exfoliating sock, in accordance with an embodiment of the invention. Referring to FIG. 3, there is shown a sock 302 with a frontal opening 306. The sock 302 comprises an upper cuff portion 304, a loop connector 308, an upper portion of the frontal opening 312 and a lower portion of the frontal opening 314.

The loop connector 308 is operable to couple an upper portion of the frontal opening 312 to the lower portion of the frontal opening 314 in order to prevent slippage and also prevent the sock 302 from rolling up on the foot. Although a single loop connector 308 is shown, the invention is not limited in this regard. Accordingly, the sock 302 may comprise a plurality of loop connectors, each of which may couple the upper portion of the frontal opening 312 to the lower portion of the frontal opening 314. In some embodiments of the invention, the loop connector 308 may comprise an elastic material. In other embodiment of the invention, the loop connector 308 may comprise a non-elastic or inelastic material. The loop connector 308 may or may not comprise an abrasive material. The loop connector 308 may be placed in the center of the socks so that socks can be worn on either foot. Notwithstanding, the invention is not limited in this regard, and the loop connector 308 may be placed at different positions along the upper portion of the frontal opening 312 and the lower portion of the frontal opening 314 of the frontal opening 306.

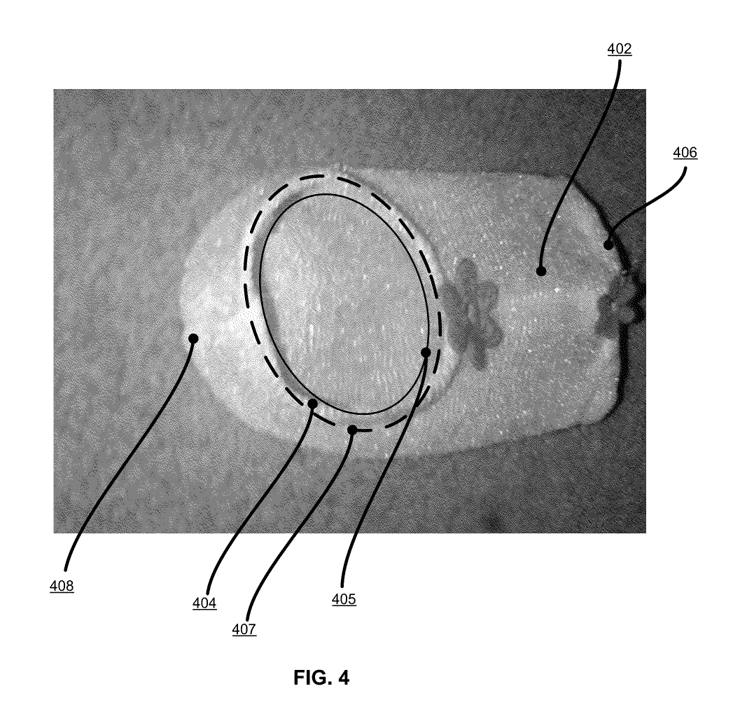

FIG. 4 is a diagram illustrating a top view of an exemplary exfoliating sock, in accordance with an embodiment of the invention. Referring to FIG. 4, there is shown a sock 402. The sock 402 comprises an upper cuff portion 404, an upper portion of the frontal opening 406, mechanism 407 and a heel portion 408. The circumferential area 405 of the upper cuff portion 404 is also illustrated.

The upper cuff portion 404 covers at least an area around an upper portion of an ankle of the human foot 200 (FIG. 2) when the sock 402 is properly placed on the human foot 200. The human foot 200 is placed within the circumferential area 405 when the sock is being placed on the human foot 200. The circumference of the upper cuff portion 404 may comprise a mechanism 407 such as a string and/or an elastic material that enables the upper cuff portion 404 of the sock to fit snugly around the human foot 200. Other mechanisms and/or materials may be utilized without departing from the spirit and scope of the various embodiments of the invention.

The sock 402 may activate the skin's peeling effect causing the outer layer of the skin to peel off, thereby removing build up, rough spots, and calluses and promotes softer, more youthful looking feet. The sock 402 may be operable to engage the process of exfoliating the human feet by gently and safely removing the dead skin cells off of the outermost layer of skin in order to reveal newer skin below. The sock 402 may leave the skin primed for moisturizing after it is utilized without using harsh chemicals or mechanical devices. The sock 402 may safely and naturally flake off or remove build up on the user's foot by using our natural circular movements and does not require the user to bend over and rub their feet. The sock 402 may be used wet or dry and is non-invasive. The sock 402 may scale off build up, rough spots and/or calluses off of the users foot while massaging the foot, aiding in increased circulation and blood flow. The sock 402 may also improve the skin tone of the user by revealing new skin beneath.

It should be recognized that although the foot covering comprises a sock 402, the invention is not limited in this regard. Accordingly, other embodiments of the invention may differ from the sock 402. For example, the sock 402 may comprise a fastener that extends from the front of the upper cuff portion 404 to the top of the frontal opening 406 (an upper portion of the frontal opening 406 of FIG. 4). In this regard, the top of the sock 402 may comprise a left upper portion and a right upper portion, which may be secured together utilizing the fastener. The fastener may comprise, for example, a string, lace, snaps, a zipper, Velcro or other fastening or securing mechanism. The fastener may be placed at any angle as long as the fastener extends from the front of the upper cuff portion 404 to the upper portion of the frontal opening 406.

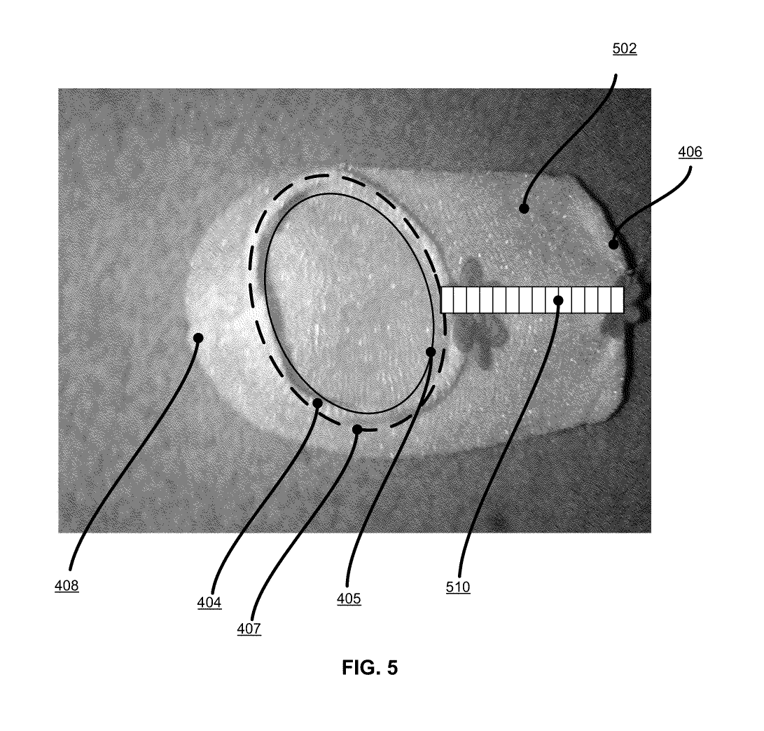

FIG. 5 is a diagram illustrating a top view of an exemplary exfoliating sock comprising a fastener, in accordance with an embodiment of the invention. Referring to FIG. 5, there is shown a sock 502. The sock 502 of FIG. 5 is substantially similar to the sock 402 of FIG. 4. The sock 502 comprises an upper cuff portion 404, an upper portion of the frontal opening 406, mechanism 407, a heel portion 408 and a fastener 510. The circumferential area 405 of the upper cuff portion 404 is also illustrated. The fastener 510 may run from the front of the circumferential area 405 of the upper cuff portion 404 straight to the upper portion of the frontal opening 406.

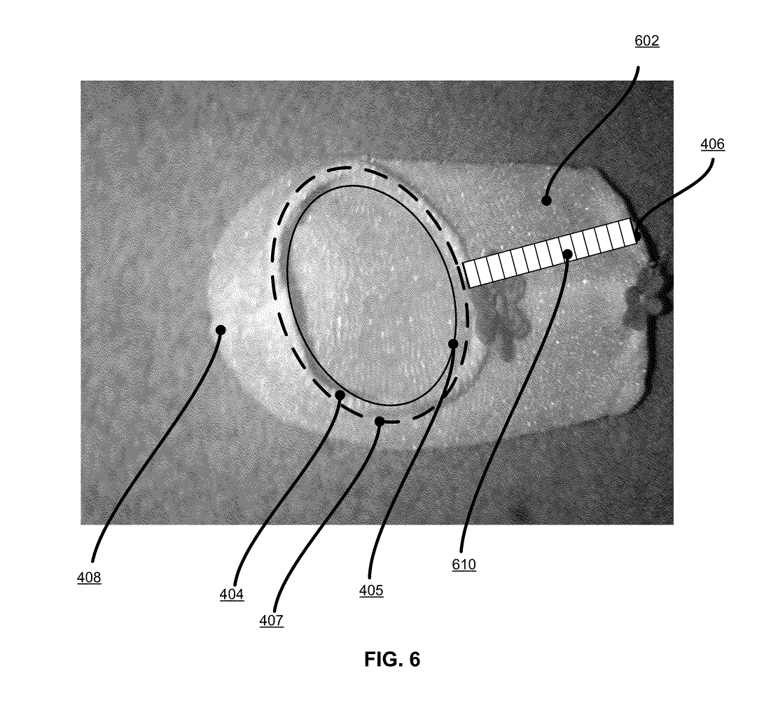

FIG. 6 is a diagram illustrating a top view of an exemplary exfoliating sock comprising an angled fastener, in accordance with an embodiment of the invention. Referring to FIG. 6, there is shown a sock 602. The sock 602 of FIG. 6 is substantially similar to the sock 402 of FIG. 4. The sock 602 comprises an upper cuff portion 404, an upper portion of the frontal opening 406, mechanism 407, a heel portion 408 and a fastener 610. The circumferential area 405 of the upper cuff portion 404 is also illustrated. The fastener 510 may run from the front of the circumferential area 405 of the upper cuff portion 404 at an angle to the upper portion of the frontal opening 406.

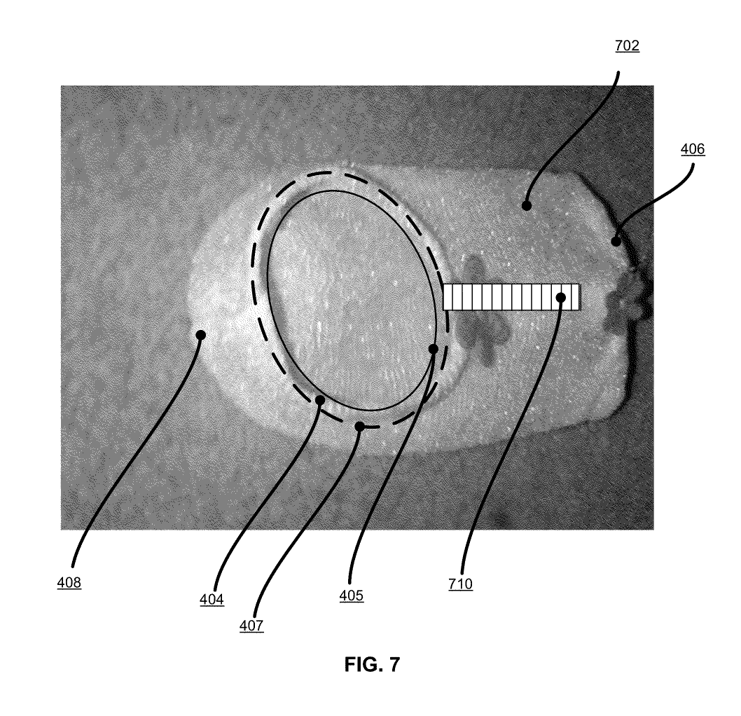

FIG. 7 is a diagram illustrating a top view of an exemplary exfoliating sock comprising a fastener, in accordance with an embodiment of the invention. Referring to FIG. 7, there is shown a sock 702. The sock 702 of FIG. 7 is substantially similar to the sock 402 of FIG. 4. The sock 702 comprises an upper cuff portion 404, an upper portion of the frontal opening 406, mechanism 407, a heel portion 408 and a fastener 710. The circumferential area 405 of the upper cuff portion 404 is also illustrated. The fastener 710 may run from the front of the circumferential area 405 of the upper cuff portion 404 partially to the upper portion of the frontal opening 406, but does not extent all the way to the upper portion of the frontal opening 406.

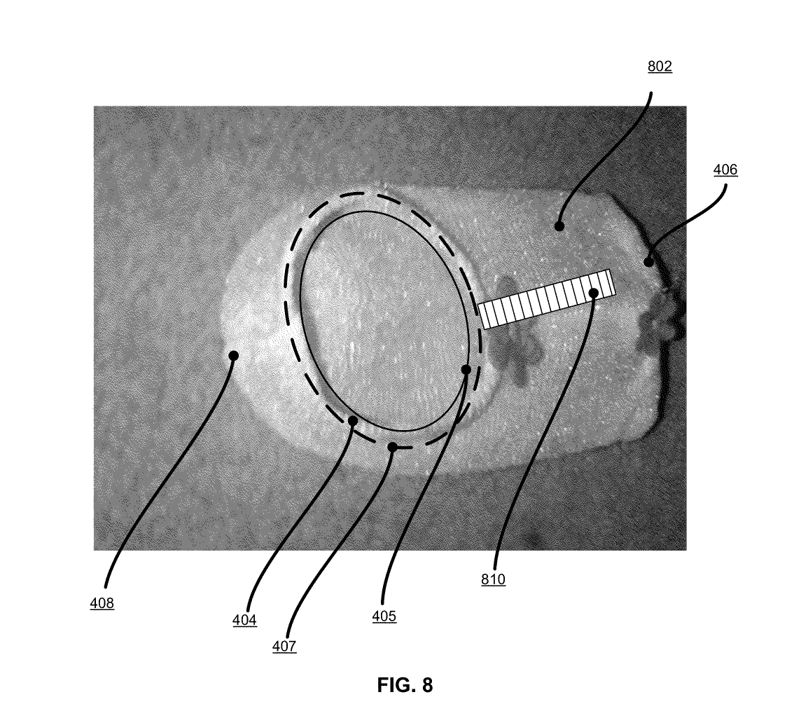

FIG. 8 is a diagram illustrating a top view of an exemplary exfoliating sock comprising an angled fastener, in accordance with an embodiment of the invention. Referring to FIG. 8, there is shown a sock 802. The sock 802 of FIG. 8 is substantially similar to the sock 402 of FIG. 4. The sock 802 comprises an upper cuff portion 404, an upper portion of the frontal opening 406, mechanism 407, a heel portion 408 and a fastener 810. The circumferential area 405 of the upper cuff portion 404 is also illustrated. The fastener 810 may run from the front of the circumferential area 405 of the upper cuff portion 404 at an angle partially to the upper portion of the frontal opening 406, but does not extent all the way to the upper portion of the frontal opening 406.

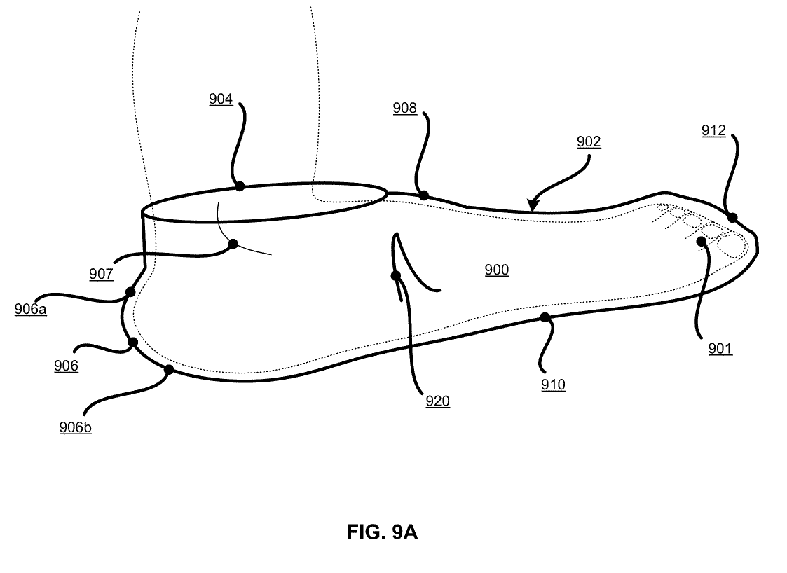

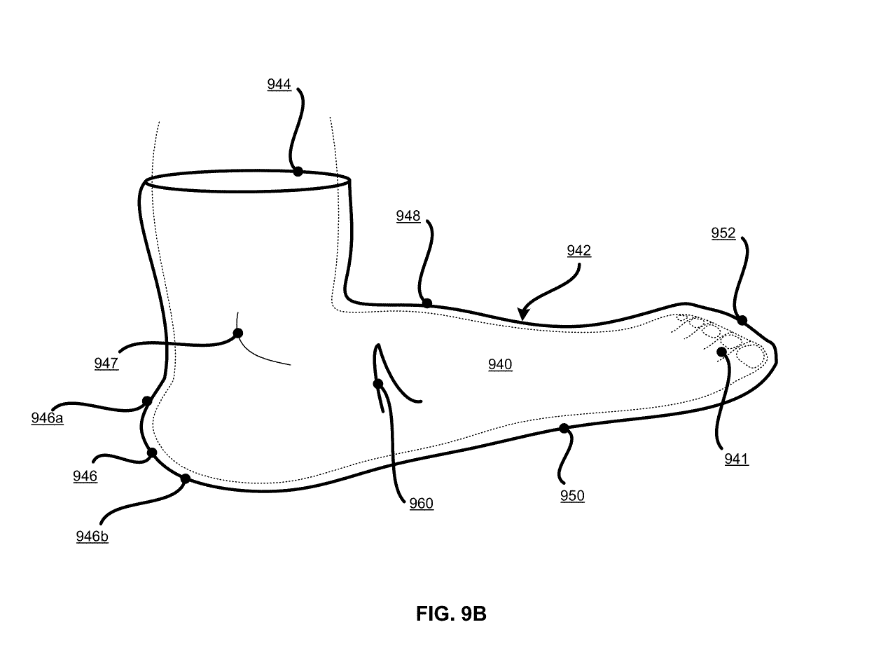

FIG. 9A is a diagram illustrating a side view of an exemplary exfoliating sock that covers the toes, in accordance with an embodiment of the invention. Referring to FIG. 9A, there is shown a sock 902 being worn on a human foot 900 with the toes 901 of the human foot being covered by the sock 902. The sock 902 comprises an upper cuff portion 904, a heel portion 906, an instep portion 908, a sole portion 910, a toe portion 912 and a gusset 920. The heel portion 906 also comprises a heel flap portion 906a and a heel turn portion 906b. FIG. 9A also shows the ankle 907 of the human foot 900.

The sock 902 may comprise an opening at the upper cuff portion 904 and top of the back heel section in order for the wearer of the sock 902 to easily insert the human foot 900 into the sock 902 and encase the human foot 900 from the heel portion 906 to the toe portion 912. When compared to the sock 202, which is illustrated in FIG. 2, the sock 902 of FIG. 9A has no frontal opening 212. Accordingly, the toes 901 on the human foot 900 of FIG. 9A are covered by the sock 902. The area around the heel 906 extends upwards towards the anklebone and slightly cascades down on the top of the human foot 900 to enable the wearer to easily insert the human foot 900 into the sock 902. The body of the sock 902, which may also comprise the instep portion 908, may comprise elastic qualities from the heel portion 906 toward the toe portion 912 so that most wearers regardless of their foot size may wear the sock 902. The body of the sock 902 comprises a toe portion 912 that ends at the tip of the toes 901 of the human foot 900. The toe portion 912 may comprise a material that prevents decorations on the toenails such as toenail polish from being damaged or smudged due to the material of the inner portion of the toe portion 912 of the sock 902 rubbing against the decorations on the toenails.

The upper cuff portion 904 is the uppermost part of the sock 902. The human foot 900 is inserted into a circumferential opening of the upper cuff portion 904 of the sock 902 and the material of the sock 902 wraps around the left, right and back side of the ankle of the human foot 900. The upper cuff portion 904 has elasticity to stretch the sock 902 around the wearer's ankle 907 as well as to hold the sock 902 in place on the wearer's foot. The upper cuff portion 904 cascades down the center of the top of the wearer's foot in, for example, a triangular manner and may comprise a ribbing around the upper cuff portion 904, which may, for example, be less than 1 inch in diameter.

The upper cuff portion 904 may cover at least an area in the vicinity of the ankle 907 of the human foot 900 when the sock 902 is properly placed on the human foot 900. The elasticity of the upper cuff portion 904 of the sock 902 enables the sock 902 to fit snuggly around the portions of the human foot 900, which are covered by the sock 902. The circumference of the upper cuff portion 904 may comprise a mechanism such as a string and/or an elastic material that enables the upper cuff portion 904 of the sock to fit snugly around the human foot 900. Other mechanisms and/or materials may be utilized without departing from the spirit and scope of the various embodiments of the invention.

The heel portion 906 may cover at least an area around a heel of the human foot 900 when the sock 902 is properly placed on the human foot 900. The heel portion 906 may comprise a heel flap portion 906a and a heel turn portion 906b. The heel flap portion 906a and the heel turn portion 906b may be substantially similar to the heel flap portion 206a and the heel turn 206b portion, which are illustrated in FIG. 2.

The heel flap portion 906a comprises the area of the sock 902 that covers the heel 906 when the sock 902 is properly fitted on the human foot 900. The heel flap portion 906a may comprise a very strong material in order to prevent this area of the sock 902 from being worn out. In this regard, the heel flap portion 906a may comprise a thicker material than other parts of the sock 902. The heel flap portion 906a may comprise one area where a great deal of exfoliation occurs.

The heel turn 906b portion is the part of the sock 902 that cradles the heel 906 and bottom of the foot when the sock 902 is properly fitted on the human foot 900. The heel turn 906b portion comprises a very strong material in order to prevent this area of the sock 902 from being worn out. In this regard, the heel turn portion 906b may comprise a thicker material than other parts of the sock 902. The heel turn portion 906b may comprise another area where a great deal of exfoliation occurs

The instep portion 908 forms part of the body of the sock 902. The instep portion 908 of the sock 902 may be viewed as the part of the sock 902 starting from where the gusset 920 ends at the area before where the toes 901 begins. The instep portion 908 covers at least an area around an instep of the human foot 900 when the sock 902 is properly placed on the human foot 900. The length of the instep portion 908 may be generally determined by measuring the wearer's foot from the back of the heel 902 to the tip of the toe portion 912. The instep portion 908 may comprise elastic properties.

The sole portion 910 covers at least an area around a sole of the human foot 900 when the sock 902 is properly placed on the human foot 900.

The toe portion 912 of the sock 902 causes the toes 901 of the human foot 900 to be covered when the sock 902 is properly placed on the human foot 900. The toe portion 912 may comprise material that enables the front portion of the sock 900 to cover and fit snugly around the toes 901 of the human foot 900. The material inside the toe portion 912 of the sock 902, which is in contact with the toes 901, may comprise a material that is non-abrasive and/or soft so that it does not in any way damage the natural state of the toenails and/or any decorations and/or material, which may be on the toenails.

The gusset 920 connects the heel flap portion 906a and heel turn portion 906b of the sock 902 to the foot of the sock 902. The gusset 920 goes from the right side of the human foot 900, around the sole 910, and up the left side of the foot 900. The gusset 920 does not go across the top of the human foot 900 because the upper cuff portion 904 extends down slightly on the top of the human foot 200.

In operation, an inner material or surface of the sock 902 may cause the skin on portions of the human foot 900, which are covered by the sock 902, to be exfoliated while the sock 902 is being worn on the human foot 900. In one embodiment of the invention, the inner material of the sock 902 may comprise the same grade of abrasiveness throughout the sock 902, except for the toe portion 912. In another embodiment of the invention, different portions of the inner material of the sock 902 may comprise varying grades of abrasiveness, with the toe portion 912 having the least or no abrasive character. In this regard, material with greater abrasiveness may be placed at those areas of the sock 902 where the foot 900 may need more exfoliation and material with lesser abrasiveness may be placed at those areas of the sock 902 where the foot 900 may need less exfoliation. The area of the toe portion 912 in contact with the toes 901 has the least or no abrasive character so as not to damage the natural state of the toenails and/or any decorations and/or material, which may be on the toenails.

In some areas of the sock 902, the material may be raised and/or contoured to match the contour of the human foot 900. The inner material of the sock 902 is operable to gently move against the skin on those portions of the human foot 900, which are covered by the sock 902, in order to cause the skin to be exfoliated. The material that is utilized to make the sock 900 possesses one or more exemplary properties comprising breathable, antifungal, antibacterial, lightweight, elastic, porous material and quick drying.

FIG. 9B is a diagram illustrating a side view of an exemplary exfoliating sock that covers the toes, in accordance with an embodiment of the invention. Referring to FIG. 9B, there is shown a sock 942 being worn on a human foot 940 with the toes 941 of the human foot being covered by the sock 942. The sock 942 comprises an upper cuff portion 944, a heel portion 946, an instep portion 948, a sole portion 950, a gusset 960 and a toe portion 952. FIG. 9B also shows the ankle 947 of the human foot 940. The heel portion 946 may comprise a heel flap portion 946a and a heel turn portion 946b. The upper cuff portion 944, the heel portion 946, the instep portion 948, the sole portion 950, the heel flap portion 946a and the heel turn portion 946b may be substantially similar to the corresponding components which are shown in and described with respect to FIG. 9A, except that the upper cuff portion 944 is above the ankle 947 of the human foot 940.

The upper cuff portion 944 is the uppermost part of the sock 942. The human foot 940 is inserted into circumferential opening of the upper cuff portion 944 of the sock 942 and the material of the sock 942 wraps around the left, right, front and back side of the ankle 947 of the human foot 940. The upper cuff portion 944 has elasticity to stretch the sock 942 around the wearer's ankle 947 as well as to hold the sock 942 in place on the wearer's foot. The upper cuff portion 944 cascades down the center of the top of the wearer's foot in, for example, a triangular manner and may comprise a ribbing around the upper cuff portion 944, which may, for example, be less than 1 inch in diameter.

The upper cuff portion 944 may cover at least an area in the region of the ankle 947 of the human foot 940 when the sock 942 is properly placed on the human foot 940. The elasticity of the upper cuff portion 944 of the sock 942 enables the sock 942 to fit snuggly around the portions of the human foot 940, which are covered by the sock 942. The circumference of the upper cuff portion 944 may comprise a mechanism such as a string and/or an elastic material that enables the upper cuff portion 944 of the sock to fit snugly around the human foot 940. Other mechanisms and/or materials may be utilized without departing from the spirit and scope of the various embodiments of the invention.

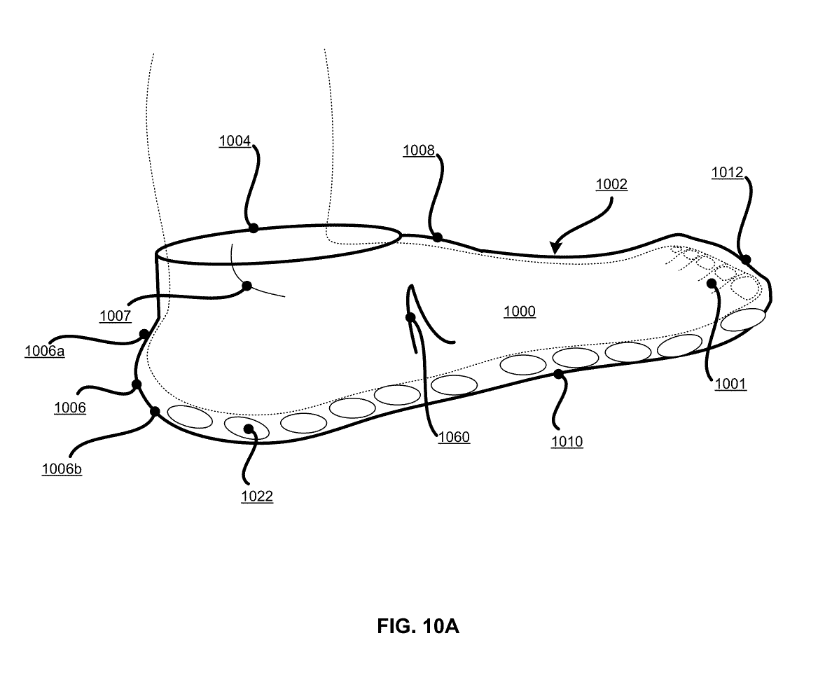

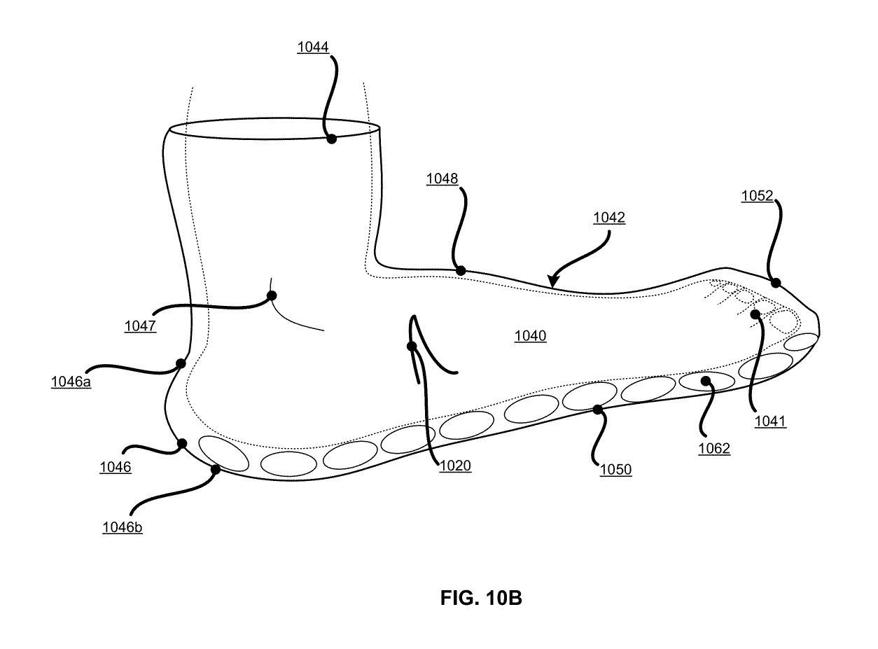

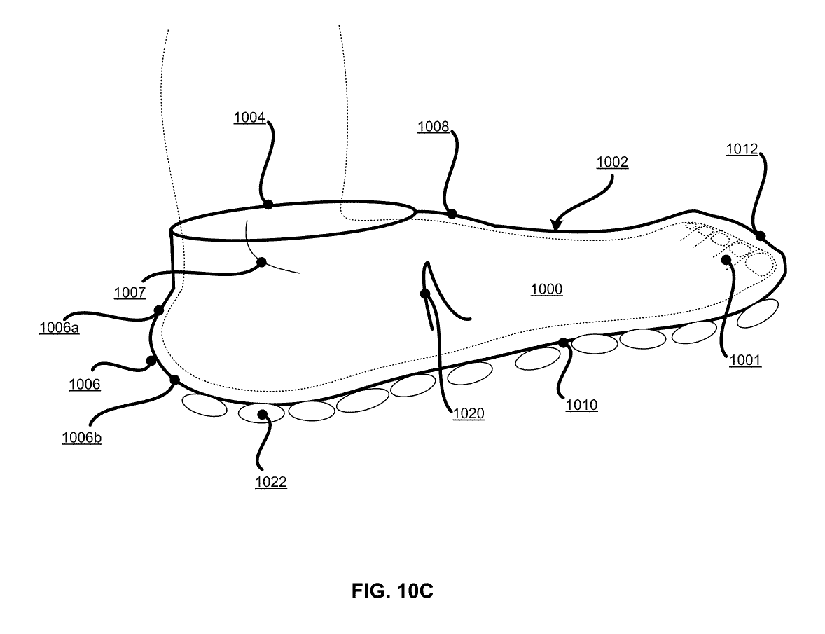

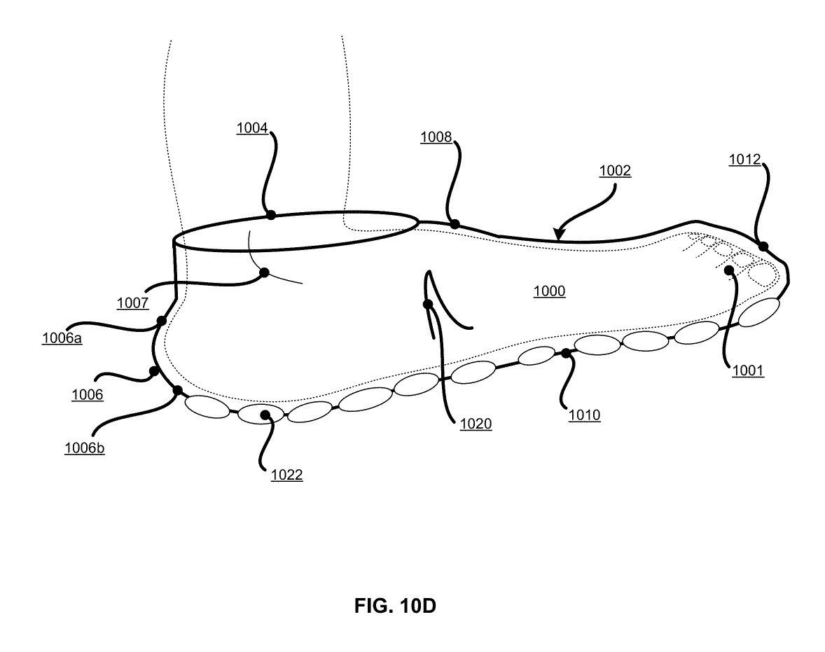

FIG. 10A is a diagram illustrating a side view of an exemplary exfoliating sock that covers the toes and comprises massaging elements, in accordance with an embodiment of the invention. Referring to FIG. 10A, there is shown a sock 1002 being worn on a human foot 1000 with the toes 1001 of the human foot being covered by the sock 1002. The sock 1002 comprises an upper cuff portion 1004, a heel portion 1006, an instep portion 1008, a sole portion 1010, a toe portion 1012, a gusset 1020 and a plurality of massaging elements 1022. The heel portion 1006 also comprises a heel flap portion 1006a and a heel turn portion 1006b. The sock 1002 of FIG. 9A also shows the ankle 1007 of the human foot 1000. The upper cuff portion 1004, the heel portion 1006, the instep portion 1008, the sole portion 1010, the toe portion 1012, the gusset 1020, the heel flap portion 1006a and the heel turn portion 1006b may be substantially similar to the corresponding components which are shown in and described with respect to FIG. 9A, except that there are a plurality of massaging elements 1022 in FIG. 10A.

In accordance with an embodiment of the invention, the plurality of massaging elements 1022 may comprise, for example, knobs that are like little rounded knitted lumps that massage the human foot 1000 when pressure is placed on them. The massaging caused by the massaging elements 1022 may, for example, help to relieve foot fatigue, increase blood circulation, and/or relieve aches in the foot such as in the sole of the foot. The massaging elements 1022 may be placed on the interior of the sock 1002 and/or on the exterior of the sock 1002. For example, the massaging elements 1022 may be sewn or fastened into the interior of the sock 1002 on the sole portion, on the bottom of the sock 1002, on the side of the sock 1002 and/or on other portions of the sock 1002. When the massaging elements 1022 are sewn on the interior of the sock 1002, the massaging elements 1022 may press directly against the sole 1010 of the human foot 1000 and massages the human foot 1000. When the massaging elements 1022 are sewn on the interior side of the sock 1002, the massaging elements 1022 may press directly against the side of the human foot 1000 and massages the side of the human foot 1000. When the massaging elements 1022 are sewn on the exterior of the sock 1002, the massaging elements 1022 may press directly against any surface that the wearer of the sock 1002 may be in contact with and indirectly massages foot of the wearer of the sock 1002. The sock 1002 with massaging elements 1022 may be great for people with poor circulation who stand on their feet for a long time and want to relieve foot fatigue.

The plurality of massaging elements 1022 may comprise any type of solid, liquid, gas, gel and/or other type of material that may provide a massaging action as the massaging elements 1022 move against the various portions of the human foot 1000. The massaging elements 1022 may comprise balls, beads, lumps, knobs and/or any other type of formation that may provide a massaging action as the massaging elements 1022 move against the various portions of the human foot 1000. The massaging elements 1022 may also comprise indentations and/or protrusions. The massaging elements 1022 may be on a single material or on a plurality of material segments.

In some embodiments of the invention, the massaging elements may comprise a magnetic material or a magnetic coating that may be operable to provide magnetic therapy or magnotherapy.

In FIG. 10A, although the massaging elements 1022 are located on the interior of the sock 1002, the invention is not limited in this regard. Accordingly, the massaging elements 1022 may be placed on any portion of the interior of the sock 1002 and/or on any portion of the exterior of the sock 1002. For example, the massaging elements 1022 may be placed on the bottom, sides, top, front and/or back of the sock 1002

FIG. 10B is a diagram illustrating a side view of an exemplary exfoliating sock that covers the toes and comprises massaging elements, in accordance with an embodiment of the invention. Referring to FIG. 10B, there is shown a sock 1042 being worn on a human foot 1040 with the toes 1041 of the human foot being covered by the sock 1042. The sock 1042 comprises an upper cuff portion 1044, a heel portion 1046, an instep portion 1048, a sole portion 1050, a toe portion 1052, a gusset 1060 and a plurality of massaging elements 1062. FIG. 10B also shows the ankle 1047 of the human foot 1040. The heel portion 1046 may comprise a heel flap portion 1046a and a heel turn portion 1046b. The upper cuff portion 1044, the heel portion 1046, the instep portion 1048, the sole portion 1050, the heel flap portion 1046a, the heel turn portion 1046b and the plurality of massaging elements 1062 may be substantially similar to the corresponding components which are shown in and described with respect to FIG. 10A, except that the upper cuff portion 1044 is above the ankle 1047 of the human foot 1040.

In FIG. 10B, although the massaging elements 1022 are located on the interior of the sock 1002, the invention is not limited in this regard. Accordingly, the massaging elements 1022 may be placed on any portion of the interior of the sock 1002 and/or on any portion of the exterior of the sock 1002. For example, the massaging elements 1022 may be placed on the bottom, sides, top, front and/or back of the sock 1002

FIG. 10C is a diagram illustrating a side view of an exemplary exfoliating sock that covers the toes and comprises massaging elements, in accordance with an embodiment of the invention. Referring to FIG. 10C, there is shown a sock 1002 being worn on a human foot 1000 with the toes 1001 of the human foot being covered by the sock 1002. The sock 1002 comprises an upper cuff portion 1004, a heel portion 1006, an instep portion 1008, a sole portion 1010, a toe portion 1012, a gusset 1020 and a plurality of massaging elements 1022. The heel portion 1006 also comprises a heel flap portion 1006a and a heel turn portion 1006b. The sock 1002 of FIG. 10C also shows the ankle 1007 of the human foot 1000. The upper cuff portion 1004, the heel portion 1006, the instep portion 1008, the sole portion 1010, the toe portion 1012, the gusset 1020, the heel flap portion 1006a and the heel turn portion 1006b may be substantially similar to the corresponding components which are shown in and described with respect to FIG. 9A, except that there are a plurality of massaging elements 1022 in FIG. 10C.

In FIG. 10C, although the massaging elements 1022 are located on the exterior of the sock 1002, the invention is not limited in this regard. Accordingly, the massaging elements 1022 may be placed on any portion of exterior of the sock 1002.

FIG. 10D is a diagram illustrating a side view of an exemplary exfoliating sock that covers the toes and comprises massaging elements, in accordance with an embodiment of the invention. Referring to FIG. 10D, there is shown a sock 1002 being worn on a human foot 1000 with the toes 1001 of the human foot being covered by the sock 1002. The sock 1002 comprises an upper cuff portion 1004, a heel portion 1006, an instep portion 1008, a sole portion 1010, a toe portion 1012, a gusset 1020 and a plurality of massaging elements 1022. The heel portion 1006 also comprises a heel flap portion 1006a and a heel turn portion 1006b. The sock 1002 of FIG. 10D also shows the ankle 1007 of the human foot 1000. The upper cuff portion 1004, the heel portion 1006, the instep portion 1008, the sole portion 1010, the toe portion 1012, the gusset 1020, the heel flap portion 1006a and the heel turn portion 1006b may be substantially similar to the corresponding components which are shown in and described with respect to FIG. 9A, except that there are a plurality of massaging elements 1022 in FIG. 10D.

In FIG. 10D, the massaging elements 1022 are located so that they protrude partially into the exterior of the sock 1002 and partially into the interior of the sock 1002. The massaging elements 1022 may be placed on any portion of the interior of the sock 1002 and/or on any portion of the exterior of the sock 1002 so that they protrude partially into the interior of the sock 1002 and partially into the exterior of the sock 1002.

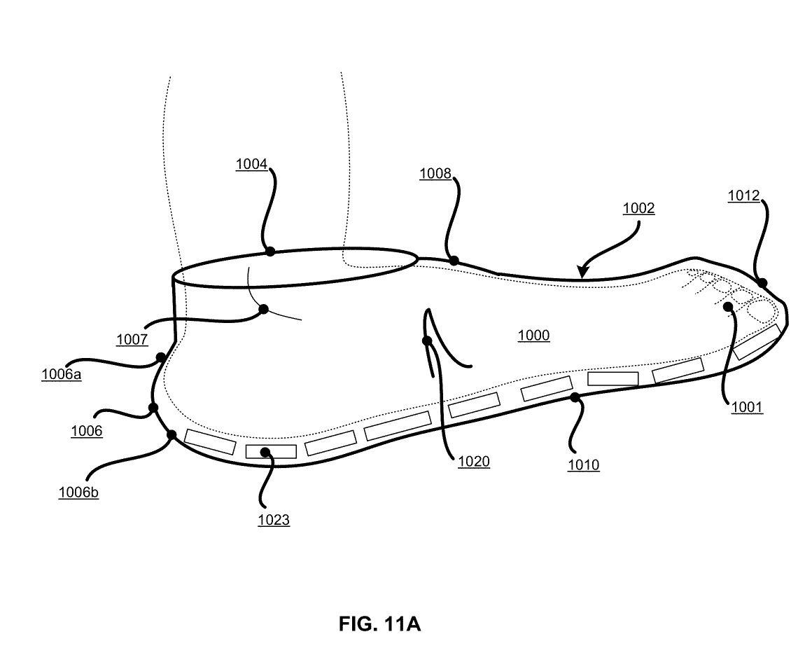

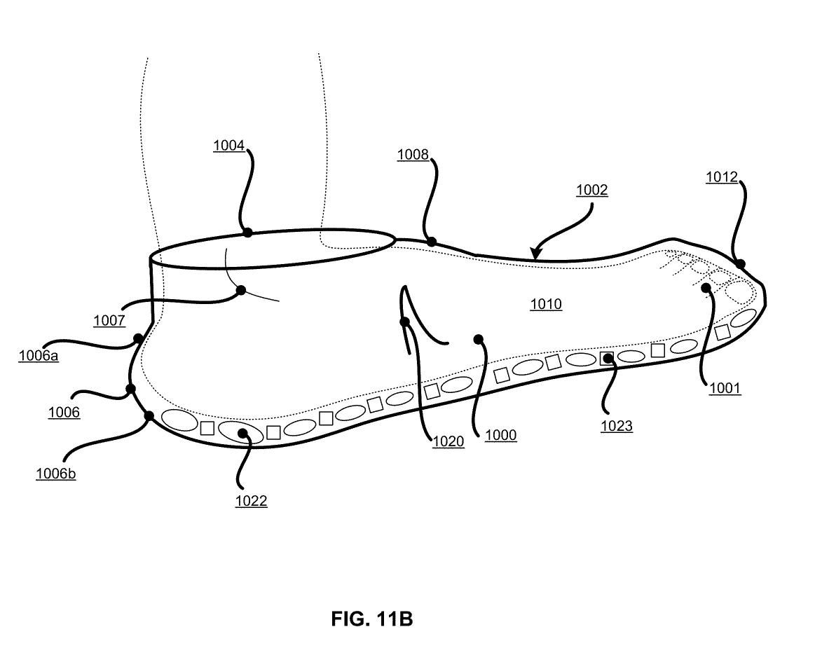

FIG. 11A is a diagram illustrating a side view of an exemplary exfoliating sock that covers the toes and comprises massaging elements, in accordance with an embodiment of the invention. Referring to FIG. 11A, there is shown a sock 1002 being worn on a human foot 1000 with the toes 1001 of the human foot being covered by the sock 1002. The sock 1002 comprises an upper cuff portion 1004, a heel portion 1006, an instep portion 1008, a sole portion 1010, a toe portion 1012, a gusset 1020 and a plurality of magnetic elements 1023. The heel portion 1006 also comprises a heel flap portion 1006a and a heel turn portion 1006b. The sock 1002 of FIG. 11A also shows the ankle 1007 of the human foot 1000. The upper cuff portion 1004, the heel portion 1006, the instep portion 1008, the sole portion 1010, the toe portion 1012, the gusset 1020, the heel flap portion 1006a and the heel turn portion 1006b may be substantially similar to the corresponding components which are shown in and described with respect to FIG. 9A, except that there are a plurality of magnetic elements 1023 in FIG. 11A.

The magnetic elements 1023 are operable to provide the therapeutic benefits of magnets to the human foot 1000 while the sock 1002 is being worn. Although in FIG. 11A the magnetic elements 1023 are shown on the interior of the sock 1002, the invention is not limited in this regard. Accordingly, the magnetic elements 1023 may be placed on any portion of the interior of the sock 1002 and/or on any portion of the exterior of the sock 1002. For example, the magnetic elements 1023 may be placed on the bottom, sides, top, front and/or back of the sock 1002

FIG. 11B is a diagram illustrating a side view of an exemplary exfoliating sock that covers the toes and comprises massaging elements, in accordance with an embodiment of the invention. Referring to FIG. 11B, there is shown a sock 1002 being worn on a human foot 1000 with the toes 1001 of the human foot being covered by the sock 1002. The sock 1002 comprises an upper cuff portion 1004, a heel portion 1006, an instep portion 1008, a sole portion 1010, a toe portion 1012, a gusset 1020, a plurality of massaging elements 1022 and a plurality of magnetic elements 1023. The heel portion 1006 also comprises a heel flap portion 1006a and a heel turn portion 1006b. The sock 1002 of FIG. 11B also shows the ankle 1007 of the human foot 1000. The upper cuff portion 1004, the heel portion 1006, the instep portion 1008, the sole portion 1010, the toe portion 1012, the gusset 1020, the heel flap portion 1006a and the heel turn portion 1006b may be substantially similar to the corresponding components which are shown in and described with respect to FIG. 9A, except that there are a plurality of massaging elements 1022 and a plurality of magnetic elements 1023 in FIG. 11B.

The massaging elements 1022 of FIG. 11B are substantially similar to the magnetic elements 1002, which are illustrated in and described with respect to FIG. 10A.

The magnetic elements 1023 are operable to provide the therapeutic benefits of magnets to the human foot 1000 while the sock 1002 is being worn.

The magnetic elements 1023 are operable to provide the therapeutic benefits of magnets to the human foot 1000 while the massaging elements 1022 concurrently massage the human foot 1000 when the sock 1002 is being worn. Although in FIG. 11B the magnetic elements 1023 and the massaging elements 1022 are shown on the interior of the sock 1002, the invention is not limited in this regard. Accordingly, the magnetic elements 1023 and/or the massaging elements 1022 may be placed on any portion of the interior of the sock 1002 and/or on any portion of the exterior of the sock 1002. For example, the massaging elements 1022 and/or the magnetic elements 1023 may be placed on the bottom, sides, top, front and/or back of the sock 1002.

In accordance with various embodiment of the invention, the massaging elements 1022 and the magnetic elements 1023 may be arranged and/or designed so that they do not hinder the exfoliating effect of the sock 1002. For example, the massaging elements 1022 may be shaped and/or spaced so that they do not interfere with the exfoliating effect of the inside of the sock 1002 as it moves against the foot 1000. Similarly, the magnetic elements 1023 may be shaped and/or spaced so that they do not interfere with the exfoliating effect of the inside of the sock 1002 as it moves against the foot 1000.

In some embodiments of the invention, the massaging elements 1022 and/or the magnetic elements 1023 may be removable elements. In this regard, the massaging elements 1022 and/or the magnetic elements 1023 may be removed from the sock 1002.

Various exemplary embodiments of the invention may comprise a foot covering to be worn on a human foot 1000. With reference to FIG. 9A comprises a sock 1002 that covers said human foot 1000. The sock 1002 comprises an inner material or surface that causes skin on portions of the human foot 1000, which are covered by the sock 1002, to be exfoliated while the sock 1002 is worn on the human foot 1000. The sock 1002 also comprises one or more massaging elements 1022 and/or one or more magnetic elements 1023 that causes the human foot 1000 to be massaged and/or be subjected to a magnetic field, respectively. Concurrent with the exfoliation, the one or more massaging elements 1022 are operable to massage at least a portion of the human foot that is within proximity of the one or more massaging elements 1022, and the one or more magnetic elements 1023 are operable to provide a magnetic field to at least a portion of the human foot that is within proximity of the one or more magnetic elements 1023.

The sock 1002 may comprise an upper cuff portion 1004 that enables the sock to fit snuggly around the portions of the human foot 1000, which are covered by said sock 1002. The upper cuff portion 1004 may comprise a string and/or an elastic material that enables the upper cuff portion 1004 of the sock 1002 to fit snugly. The one or more massaging elements 1022 and/or the one or more magnetic elements 1023 may be located along an interior or inside portion of the sock 1002 and/or an exterior or outer portion of the sock 1002. The one or more massaging elements 1022 and/or the one or more magnetic elements 1023 are located so that they protrude in an interior portion of the sock 1002 and an exterior portion of said sock 1002. The sock 1002, which covers portions of the human foot 1000, comprises an upper cuff portion 1004, a heel portion 1006, an instep portion 1008, a toe portion 1012 and a sole portion 1010. The upper cuff portion 1004 may cover at least an area within the vicinity of an ankle 1007 of the human foot. The heel portion 1006 covers at least an area around a heel of said human foot 1000. The instep portion 1008 covers at least an area around an instep of the human foot 1008. The toe portion 1012 covers at least an area around toes 1001 of the human foot 1000. The sole portion 1010 covers at least an area around a sole of the human foot 1000.

In some embodiments of the invention, the toe portion 1012 of the sock 1002 may comprise a non-abrasive material. In some embodiments of the invention, different portions of the interior or inner material of the sock 1002 may comprise varying grades of abrasiveness. The inner material of the sock 1002 is operable to gently move against the skin on the portions of the human foot 1000, which are covered by the sock 1002, to cause the skin to be exfoliated while the foot is being massaged and/or being subjected to a magnetic field. The material of the sock possesses one or more properties comprising being breathable, antifungal, antibacterial, light weight, porous material and/or quick drying.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A foot covering to be worn on a human foot, said foot covering comprising:
a sock that covers said human foot, wherein:
said sock comprises an inner material that causes skin on portions of said human foot, which are covered by said sock, to be exfoliated while said sock is worn on said human foot; and
said sock comprising one or more massaging elements and/or one or more magnetic elements, wherein:
said one or more massaging elements massaging at least a portion of said human foot that is within proximity of said one or more massaging elements; and
said one or more magnetic elements providing a magnetic field to at least a portion of said human foot that is within proximity of said one or more magnetic elements.

2. The foot covering according to claim 1, comprising an upper cuff portion that enables said sock to fit snuggly around said portions of said human foot, which are covered by said sock.

3. The foot covering according to claim 2, wherein said upper cuff portion comprises at least one of a string and an elastic material that enables said upper cuff portion of said sock to fit snugly.

4. The foot covering according to claim 1, wherein said one or more massaging elements and/or said one or more magnetic elements are located along an interior portion of said sock and/or an exterior portion of said sock.

5. The foot covering according to claim 1, wherein said one or more massaging elements and/or said one or more magnetic elements are located so that they protrude in an interior portion of said sock and an exterior portion of said sock.

6. The foot covering according to claim 1, wherein said portions of said human foot, which are covered by said sock comprises an upper cuff portion, a heel portion, an instep portion, a toe portion and a sole portion, wherein:
said upper cuff portion covers at least an upper area of an ankle of said human foot;
said heel portion covers at least an area around a heel of said human foot;
said instep portion covers at least an area around an instep of said human foot;
said toe portion covers at least an area around toes of said human foot; and
said sole portion covers at least an area around a sole of said human foot.

7. The foot covering according to claim 6, wherein said toe portion of said sock comprise a non-abrasive material.

8. The foot covering according to claim 1, wherein different portions of said inner material of said sock comprise varying grades of abrasiveness.

9. The foot covering according to claim 1, wherein said inner material of said sock gently moves against said skin on said portions of said human foot, which are covered by said sock, to cause said skin to be exfoliated.

10. The foot covering according to claim 1, wherein a material of said sock possesses one or more properties comprising breathable, antifungal, antibacterial, light weight, porous material and quick drying.

11. A method, comprising:
exfoliating a human foot utilizing a foot covering that is worn on said human foot, wherein said foot covering comprises:
a sock that covers said human foot, wherein:
said sock comprises an inner material that causes skin on portions of said human foot, which are covered by said sock, to be exfoliated while said sock is worn on said human foot; and
said sock comprises one or more massaging elements and/or one or more magnetic elements, wherein:
said one or more massaging elements enables massaging at least a portion of said human foot that is within proximity of said one or more massaging elements; and
said one or more magnetic elements providing a magnetic field to at least a portion of said human foot that is within proximity of said one or more magnetic elements

12. The method according to claim 11, wherein an upper cuff portion that enables said sock to fit snuggly around said portions of said human foot, which are covered by said sock.

13. The method according to claim 12, wherein said upper cuff portion comprises at least one of a string and an elastic material that enables said upper cuff portion of said sock to fit snugly.

14. The method according to claim 11, wherein said one or more massaging elements and/or said one or more magnetic elements are located along an interior portion of said sock and/or an exterior portion of said sock.

15. The method according to claim 11, wherein said one or more massaging elements and/or said one or more magnetic elements are located so that they protrude in an interior portion of said sock and an exterior portion of said sock.

16. The method according to claim 11, wherein said portions of said human foot, which are covered by said sock comprises an upper cuff portion, a heel portion, an instep portion, a toe portion and a sole portion, wherein:
said upper cuff portion covers at least an upper area of an ankle of said human foot;
said heel portion covers at least an area around a heel of said human foot;
said instep portion covers at least an area around an instep of said human foot;
said toe portion covers at least an area around toes of said human foot; and
said sole portion covers at least an area around a sole of said human foot.

17. The method according to claim 16, wherein said toe portion of said sock comprise a non-abrasive material.

18. The method according to claim 11, wherein different portions of said inner material of said sock comprise varying grades of abrasiveness.

19. The method according to claim 11, wherein said inner material of said sock gently moves to cause said skin on said portions of said human foot, which are covered by said sock, to be exfoliated.

20. The method according to claim 11, wherein a material of said sock possesses one or more properties comprising breathable, antifungal, antibacterial, light weight, porous material and quick drying.
